(19) United States
(12) Patent Application Publication
(10) Pub. No.: US 2024/0216632 A1
(43) Pub. Date: Jul. 4, 2024

(54) APPARATUS AND METHOD FOR SELECTING POSITIVE AIRWAY PRESSURE MASK INTERFACE

(71) Applicants: Lawrence Neal, Portland, OR (US); Sudesh Banskota, Hillsboro, OR (US); Akhil Raghuram, Vancouver, WA (US)

(72) Inventors: Lawrence Neal, Portland, OR (US); Sudesh Banskota, Hillsboro, OR (US); Akhil Raghuram, Vancouver, WA (US)

(21) Appl. No.: 18/606,842

(22) Filed: Mar. 15, 2024

Related U.S. Application Data
(63) Continuation-in-part of application No. 17/242,600, filed on Apr. 28, 2021, now abandoned.
(60) Provisional application No. 63/028,351, filed on May 21, 2020.

(51) Int. Cl. A61M 16/06; G06T 7/00

(52) U.S. Cl. CPC A61M 16/0605; G06T 7/0012; A61M 2016/0661; G06T 2207/30201

(57) ABSTRACT

Embodiments of the positive airway pressure (PAP) mask fitting system and method provide a PAP mask fitting process to a specific patient that is as automatic as possible and that returns the patient the most appropriate PAP mask fit. The PAP mask fitting is done in a relatively quick manner. Once a PAP mask fitting has identified a preferred PAP mask for the patient, an appropriate PAP mask can be ordered on demand and is quickly, and possibly immediately, provided to the patient.

### PRIORITY CLAIM

This application claims priority to copending U.S. Non-provisional application Ser. No. 17/242,600, filed on Apr. 28, 2021, entitled Apparatus and Method For Selecting Positive Airway Pressure Mask Interface, which is hereby incorporated by reference in its entirety for all purposes.

### BACKGROUND OF THE INVENTION

Obstructive and central sleep apnea are highly prevalent problems. Devices that provide positive airway pressure (PAP) are the treatment of choice for these patients. Such PAP devices are also interchangeably referred to continuous positive airway pressure (CPAP) devices in the arts. A PAP system entails the patient wearing a mask interface to deliver pressurized air to act as a pressure splint to keep their breathing airway open while they sleep. Patients still have to consider numerous PAP mask options available to find a compromise between fit, style, color, shape, price and so on.

Masks for PAP use are mass produced in standardized sizes. Each patient's face is sufficiently unique as a basic form of identification, but the patient has to choose from products made for general faces that differ from person to person. It is very difficult for the patient to find out a person's unique taste, facial skeleton and one perfect mask to suit their needs. Appropriate fit of the mask has been an ongoing challenge which is a barrier to appropriate treatment of these patients. The traditional approach and model of care has been for patients to visit a Home Medical Equipment (HME) office and have an expert fit the patient with an appropriate mask. This is a high cost method and the results of fitting the patient for their PAP mask during an in-person visit are clinician dependent and prone to variability.

Recent entrants attempting to perform remote mask fittings in lieu of in-person mask fittings have looked at having a patient use standard, easily available objects such as a US Quarter Dollar coin or ruler to measure their face and determine an appropriate mask. These fittings have been performed by means of a web-based teleconferencing software (Zoom/Skype) with an expert guiding the patient, or by the patient using a web-based application guiding them through the steps. These are difficult to follow and have poor reliability.

### Bayesian Iridocorneal Scale Estimation

A method is disclosed for converting a set of relative ratios observed on images of a human face, into absolute measurements of distances, measured in millimeters, by means of the anatomical relationships between certain points on the human eyes. Specifically the system uses the distance between eyes (interpupillary distance) and the width of the iris and cornea (“white-to-white” distance) jointly as a kind of fiducial marker or calibration target. Given a set of face keypoints, the method measures distances between keypoints at the centers of the pupils and the edges of the iris. Using the selected pupil and iris keypoint distances and a prior distribution based on patient information collected in the demographic questionnaire, the method produces a Bayesian estimate of absolute distances, measured in millimeters, which are then applied to all parts of the face for input into the Supervised Machine Learning Classifier and Expert Rules systems.

### Soft Tissue Deformable Bundle Adjustment

A method is disclosed for analyzing a set of 2D camera images of the same human face from different camera angles, in order to jointly determine the 3D geometry of the face, and the positions and angles from which each camera image was taken. The method divides the face keypoints based on anatomical rules into three sets, corresponding to deformable (soft tissue), semi-deformable, and non-deformable (bony or cartilaginous tissue). This anatomical distinction is translated into a numerical score applied to each point during a bundle adjustment optimization, in order to account for error that would otherwise be introduced by changes in facial expression.

Accordingly, in the arts of PAP system, and in particular PAP masks, there is a need for an improved process to fit a user with a PAP mask that best suits the user's needs and unique facial attributes

### SUMMARY OF THE INVENTION

Embodiments of the positive airway pressure (PAP) mask fitting system and method provide a PAP mask fitting process to a specific patient that is as automatic as possible and that returns the patient the most appropriate PAP mask fit. The PAP mask fitting is done in a relatively quick manner. Once a PAP mask fitting has identified a preferred PAP mask for the patient, an appropriate PAP mask can be ordered on demand and is quickly, and possibly immediately, provided to the patient.

Further, with the ongoing COVID19 pandemic, in-office mask fitting is deemed a risky procedure to both patients (user) and clinicians. The ability for patients to get fit with an appropriate mask at home using their smartphone or other image capture device is facilitated using embodiments of the PAP mask fitting system and method.

It is well known in the field of computer vision that any reconstruction of 3D points using 2D images is accurate only up to an unknown scale factor. That is to say, a miniature scene closer to the camera appears identical to a larger scene further away. In order to determine absolute scale, some type of calibration target or fiducial marker of a known size must be provided, against which the rest of the scene can be measured. Previous systems for face mask fitting have required an additional element, such as a U.S. coin of known size, to be included in the collected images in order to establish scale, or have used relative measurements only without establishing scale. However, when viewing images of human faces in which both eyes are visible, an estimate of scale can be made by using the specific anatomical fact that in adult humans, the diameter of the iris of the eye is 11.8 mm on average, and variations in iris diameter are generally not correlated to the rest of the facial anatomy. Using this fact and measuring the visible iris diameter and the visible interpupillary distance between the eyes, our system establishes an estimate of scale, providing an absolute interpupillary distance in millimeters. This absolute distance takes the place of a fiducial marker and enables measurement of absolute 3D distances in millimeters, which is required to select specific sizes of headgear and cushions for CPAP equipment fitting.

Synthesizing multiple 2-dimensional images into a single 3-dimensional image typically requires the use of bundle adjustment: an optimization technique in which the predicted positions of 3D points, along with the predicted positions of the cameras from which the 2D images were captured, are adjusted in order to most closely match the observed images. This technique involves solving a least-squares optimization problem to minimize a measure of error called reprojection error, based on the geometry of the predicted camera positions and 3D points. In the traditional formulation, bundle adjustment for 3D reconstruction assumes that all 3D points captured belong to a single static scene, and do not move relative to each other while the 3D capture is in progress. However, when capturing multiple images of a human face, this is often not the case: facial expressions can change during the course of a capture session, which introduces error as the deformable parts of the face (such as the lips and jaw) from some images will not match others. While processing a set of face keypoints derived from multiple images captured from a user's mobile device, we find that this error can be reduced by replacing the standard formulation of bundle adjustment with a novel, anatomically-based weighting strategy we term Soft Tissue Deformable Bundle Adjustment. Specifically, during bundle adjustment, the reprojection error is weighted such that the hard and cartilaginous parts of the face are preferentially attended to and the more deformable parts of the face are allowed to move relative to each other. This ensures proper alignment of the rigid parts of the face, especially the region between the eyes, nose, and nasal philtrum, which are most important in producing an accurate recommendation for CPAP mask fitting.

### BRIEF DESCRIPTION OF THE DRAWINGS

Advantages of the invention will be readily appreciated as the same becomes better understood by reference to the following detailed description when considered in connection with the accompanying drawings, wherein:

FIG. 1 is a combination flow diagram and block diagram of a PAP mask fitting system.

FIGS. 2 and 3 are conceptual diagrams illustrating location and vectors of selected face keypoints.

FIG. 4: Process for establishing scale based on interpupillary distance and iris diameter, with example data and result.

FIG. 5: Illustration of interpupillary distance estimation from face keypoints

FIG. 6: Illustration of iris diameter estimation from face keypoints

FIG. 7: Illustration of alignment of keypoints from multiple collected images with soft tissue deformable bundle adjustment in order to generate a single set of aligned keypoints.

FIG. 8: Illustration of the keypoints in the partially-deformable region, including the tissue around the infraorbital area above the zygomatic bone but not including the eye and eyelid.

FIG. 9: Illustration of the keypoints in the non-deformable region, including the front of the nose from the nasion to the nares.

FIG. 10 is a block diagram showing additional detail of an example PAP mask fitting system.

FIG. 11 is a schematic of the SleepGlad mask election tool data flow between the mobile device and dashboard.

### DETAILED DESCRIPTION

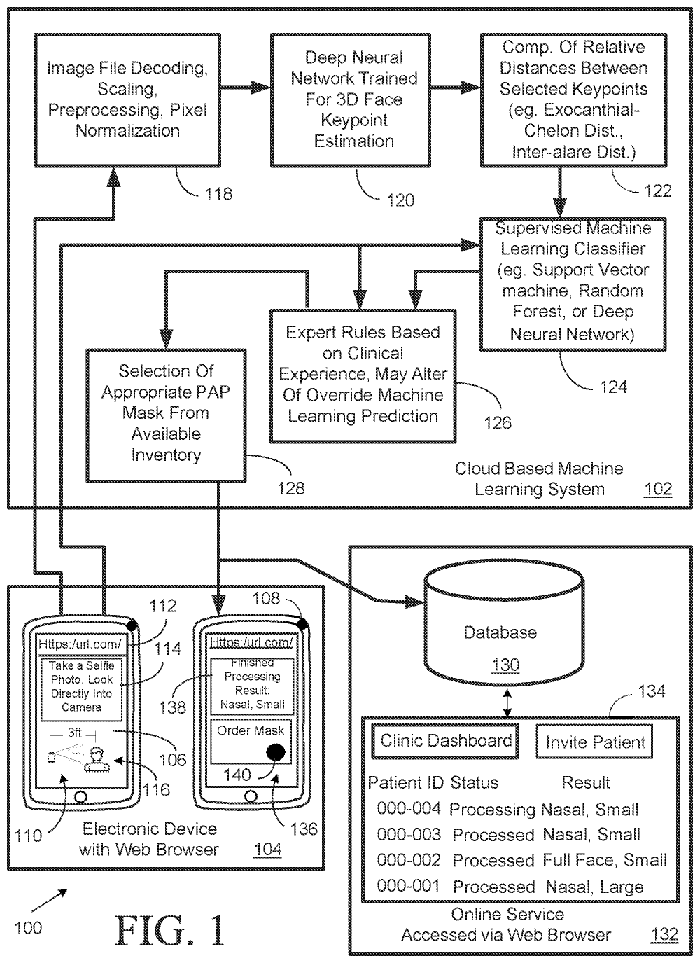

FIG. 1 illustrates an example positive airway pressure (PAP) mask fitting system 100. Embodiments of the PAP mask fitting system 100 provide a system and method for identifying selected face keypoints from a received image of the patient's face who is to be fitted for a PAP mask. Based upon characteristics of and relationships between the identified face keypoints, a particular PAP mask may be identified for a PAP patient.

The disclosed systems and methods for a PAP mask fitting system 100 will become better understood through review of the following detailed description in conjunction with the figures. The detailed description and figures provide examples of the various inventions described herein. Those skilled in the art will understand that the disclosed examples may be varied, modified, and altered without departing from the scope of the inventions described herein. Many variations are contemplated for different applications and design considerations, however, for the sake of brevity, each and every contemplated variation is not individually described in the following detailed description.

Throughout the following detailed description, a variety of examples for systems and methods for the PAP mask fitting system 100 are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional, elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

“Communicatively coupled” means that an electronic device exchanges information with another electronic device, either wirelessly or with a wire based connector, whether directly or indirectly through a communication network 108. “Controllably coupled” means that an electronic device controls operation of another electronic device.

Returning to FIG. 1, a combination flow diagram and block diagram of a PAP mask fitting system 100 is illustrated. A non-limiting embodiment of the PAP mask fitting system 100 employs a cloud based machine learning system 102 that receives image data from an electronic device 104 provisioned with a web browser and an image capture device 106.

In the hypothetical embodiment illustrated in FIG. 1, the electronic device 104 is generically illustrated as a smart phone provisioned with a display 106 and an image capture device 108 that is oriented inward so as to be configured to capture an image of the patient. The captured image of the patient is interchangeably referred to as a “selfie” herein.

Other types of electronic devices 104 may be used with embodiments of the PAP mask fitting system 100. For example, a laptop or personal computer provisioned with an image capture device (camera) may be used with the PAP mask fitting system 100. Other examples electronic devices 104 include cellular phones, notebooks, personal device assistants, or the like. The patient might even take a selfie with a legacy camera, and then email the captured image to the PAP mask fitting system 100. Any electronic device now known or later developed is intended to be within the scope of this disclosure.

To initiate operation of the PAP mask fitting system 100, the patient using their electronic device 104 initiates an interactive session with the cloud based machine learning system 102. Using a web interface, a clinical operator and/or the cloud based machine learning system 102 creates an electronic record specifying an individual patient. This record may include the patient's name, phone number, and medical information. In a non-limiting example embodiment, when the patient record is created, an SMS text message is automatically sent to the patient's mobile phone number. This text message contains an individualized message and a hyperlink 112, preferably to be opened one time only, by the patient, on their electronic device 104, such as their mobile smart phone device. The hyperlink address 112 is for a particular web site that is the portal for an interactive PAP mask fitting session. Alternatively, or additionally, the individualized message and the hyperlink 112 may be communicated to another designated electronic device.

After receiving the individualized message and the hyperlink 112, the patient logs in to a secure portal (server) of the cloud based machine learning system 102 to establish a secure interactive PAP mask fitting session. If the patient is using their smart phone 104, the patient may log in using the SMS message text. The hyperlink directs the patient to a user interface, containing personalized messaging for this patient, a set of medical questions, and an optional photo upload button (that is later used to upload a capture image of the patient's face to the cloud based machine learning system 102). The user patient answers the medical questions through the Web interface via a presented graphical user interface (GUI). Example medical questions include, but are not limited to, sleep difficulties, breathing difficulties, preferences about wearing glasses, facial hair, dental problems, and other issues that may affect the proper choice of PAP equipment.

The patient is instructed to take a photograph of their own face using their mobile device 104 or another image capture device, according to some simple instructions such as, but not limited to, “Hold the camera at arm's length, hold the camera at eye level, look directly at the camera, and take the photo.” For example, the electronic device 104 receives a GUI 110 that is presented on the display 106. The non-limiting GUI 110 presents information indicating the hyperlink address 112, textual user instructions 114 for capturing a selfie image, and/or a graphical image 116 that graphically instructs the patient. Based on the instructions, the patient captures an image of their face. Any suitable GUI, or series of GUIs, may be used to facilitate the capture of an image of the patient's face. Other alternative GUIs may present more information, less information, and/or different information to guide the patient through the interactive PAP mask fitting session.

Once the image of the patient has been acquired, the image data is communicated to the cloud based machine learning system 102. Preferably, only a single image of the patient's face is required. Alternatively, or additionally, multiple images of the patient's face may be acquired. Multiple images may be taken from different angles of the patient's face, such as a side view or the like. In some embodiments, the patient may capture a short video clip of their face from which multiple 2D images may be acquired from. In some embodiments, the video may be live streamed to the PAP mask fitting system 100 for a real time, or a near real time, PAP mask fitting process.

Various supplemental information may also be input by the patient via the presented GUIs. For example, the patient may input their name, age, sex, contact information, health provider information, location information, account information, or the like that will be used to facilitate procuring a PAP mask for the patient.

The communicated image data of the patient's face is received and decoded at block 118. The image data of the patient's face is converted to an uncompressed format, scaled, and/or cropped to the appropriate size, and normalized to a format appropriate for input to a convolutional neural network. For example, but not limited to, the image data is processed by scaling the image of the patient's face to a standard size. In some embodiments, pixel normalization may be conducted so that the pixels of the preprocessed image data corresponds to the pixel attributes of a normalized face image. Some embodiments may adjust pixel brightness, luminosity, granularity, and/or color of the received image pixel data. The pixel data may be adjusted using any suitable algorithm now known or later developed.

In a preferred embodiment, at block 120, the pixel array (the processed image data) is fed as input to a trained convolutional neural network which predicts 3D positions of face keypoints from 2D image data. In a preferred embodiment, the convolutional neural network is a deep neural network. Any suitable convolutional neural network now known or later developed may be used in the various embodiments.

The deep neural network is trained to recognize two dimensional (2D) key face points of the patient's face in the 2D processed image data. The deep neural network determines corresponding three dimensional (3D) face keypoints. In three dimensions, the determined 3D face keypoints are defined in 3D space with respect to a reference point. Here, the neural network has already been trained using a large representative dataset of human faces, not necessarily limited to PAP patients. Any suitable neural network or suitable algorithm now known or later developed that identifies the face keypoints in the received 2D image data of the patient's face to determine corresponding 3D face keypoints may be used in alternative embodiments.

The neural network outputs a set of 3D points in a pre-specified order, corresponding to the estimated spatial location of face landmark points. The points include the corners of the mouth (Chelion left and right), corners of the inside of the eyes (Endocanthion left and right), corners of the outside of the eyes (Exocanthion), outer edges of the nose (Alare), bridge of the nose (Nasion) and other key face points. Some embodiments may identify boundaries of the eye iris for each eye. The iris data may be used to, but is not limited to, defining a scale factor of the patient's head. Any suitable number of and/or types of face keypoints may be determined in 3D space by the various embodiments.

There are various keypoint identification modules that perform this task, usually used for face recognition, emotion detection, or safety tasks. Some non-limiting examples include:
a. A Face Alignment Network method.
b. A Joint Face Alignment and 3D Face Reconstruction.
c. A faster than real-time facial alignment such as 3d spatial transformer network approach in unconstrained poses.
d. A Pose-Invariant 3D Face Alignment method.
e. A “pose-invariant face alignment method.
f. Other types of convolutional networks, including generative adversarial networks, recurrent neural networks, and non-convolutional methods.

At block 122, a set of Euclidean distances between face keypoints are calculated, including the inter-alare distance (width of the nose), the chelion-to-exocanthion distance (height of the face from lips to eyes), and other relevant distances between face keypoints. Since the location information for each of the identified face keypoints is defined in 3D space, the computed distances may be represented as vectors in 3D space by some embodiments. Any suitable 3D coordinate system may be used by the various embodiments to compute these distances. Further, angles associated with each computed distance are determined to generate a vector.

At block 124, these vectors, along with the patient's answers to the web interface questions, and the medical information in the record created by the clinical operator and/or the cloud based machine learning system 102, are converted to a format appropriate for input to a supervised machine learning classifier. For example, the inputs may be converted to floating point numbers, and statistically standardized (subtracted from a predetermined mean value and divided by a predetermined standard deviation). The converted inputs are referred to as a facial feature vector.

The facial feature vector is input to a supervised machine learning classifier, which has already been trained with a pre-existing reference dataset, for the task of mask type classification. The classifier may be a Support Vector Machine, Random Forest, Logistic Regression, Deep Neural Network, or other employ another similar method. In some embodiments, the classifier may be an ensemble: a set of multiple SVM, Random Forest, etc., or a combination of such, each of which outputs an independent prediction, and whose predictions are averaged or otherwise aggregated to produce a final prediction. Each classifier predicts, given an input feature vector, which one out of a set of known CPAP mask types (full face, nasal, nasal pillows, etc.) is most likely to correctly fit the patient described by the feature vector. In some embodiments, Multi-Label Classification may be used to account for the possibility that more than one mask type may be appropriate for the patient, the supervised classifier may generate multiple predictions, one for each mask type, indicating the probability of fit. Then, a mask type and/or size prediction is computed. In addition to mask type, another classifier of the same type may be used to predict mask size, out of the set of possible sizes (small, medium, large, etc.). This size classifier may be independent of the type classifier, in which case the size and type are each predicted independently from the same facial feature vector, or the two classifiers may be integrated together, in which case a single prediction is made (e.g. Medium Nasal, Small Full-Face, etc.)

In some embodiments, demographics of the patient may be incorporated into the PAP mask fitting process. In such embodiments, members of a particular demographic category may have one or more facial and/or medical attributes that are relatively common along their demographic. Demographics may include age, sex, race, or the like of the patient undergoing the PAP mask fitting process.

At block 126, the prediction output by the supervised classifier, along with the demographic and medical information collected from the patient and input by the operator, is input to a software component that applies a set of predetermined rules based on clinical knowledge, which may augment or override the machine learning output. For example, but not limited to, if a patient has claustrophobia, then the PAP mask fitting system 100 would not recommend a full-face style mask. Given the recommended PAP mask type/size (e.g. Nasal Mask, Medium), and based on operator preferences and availability of supplies, one or more specific models of CPAP mask (e.g. Fisher & Paykel Eson 2 with Medium headgear and Medium cushion) are identified and are output at block 128.

The information identifying the recommended PAP model, after all rules have been applied, is stored in a database 130. In an example embodiment, the database 130 may reside at an online service accessed web browser 132. The PAP mask recommendations may be returned, via the Web interface 132, to both to the patient's electronic device 104 and to the clinical operator. The example data 134 may be stored in a relational database or the like that associates the user patient's identity, their processing status, and the resultant PAP mask recommendation.

In an example embodiment, a non-limiting example GUI 136 may then be presented to the patient on the display 116 of their electronic device 104. Textual information 138 indicating the PAP mask recommendation may be presented to the patient. Additionally, or alternatively, images of the recommended PAP mask (not shown) may be presented to the patient. Optionally, an active hot spot 140 on the touch sensitive display 116 of the patient's electronic device 104 may be provided to enable the patient to procure the recommended PAP mask.

Afterwards, additional follow-up communication may be sent to the patient or operator, to determine whether the recommended PAP mask was correct. This feedback information may be communicated back to the cloud based machine learning system 102 to enhance the learning of the neural network.

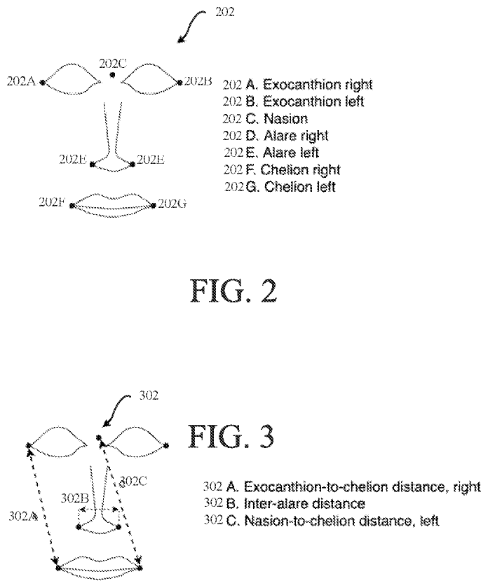

FIGS. 2 and 3 are conceptual diagrams illustrating location 202 and vector 302 between selected face keypoints. In an example embodiments, a generic human face is illustrated which shows various key face points that are determinable for a received image of the patient's face. Facial keypoints 202a and 202b correspond to the exocanthion right and the exocanthion left key face points, respectively. Facial keypoint 202c is a nasion face keypoint. Facial keypoints 202d and 202e are the alare right and alare left face keypoints, respectively. Facial keypoints 202f and 202g are the chelion right and chelion left face keypoints, respectively.

In FIG. 2, several example vectors that are computed during the PAP mask fitting process are illustrated. Vector 302a corresponds to the exocanthion-to cehlion distance vector, right. Vector 302b corresponds to the inter-alare distance vector. Vector 302c corresponds to the nasion-to-chelion distance vector, left. One skilled in the art appreciates that numerous additional vectors between selected face keypoints are determined during the PAP mask fitting process.

When a plurality of 2D images are used to determine the face keypoints in 3D space, location information of like face keypoints may be combined, averaged otherwise combined to improve the accuracy of the determined location of the patient's face keypoints.

When the distances and angles (vectors) between patient's face keypoints have been determined by the PAP mask fitting system 100, a patient's facial feature vector is determined for the patient. In an example embodiment, the patient's facial feature vector may be represented in a matrix or other suitable format.

Each PAP mask has a corresponding facial feature vector. When a patient's facial feature vector matches or corresponds with the PAP mask facial feature vector of a particular PAP mask, that PAP mask may be identified as a suitable candidate PAP mask for consideration for use by the patient. It is likely that for any particular patient, a plurality of different PAP masks may be identified as candidate PAP masks.

Preferably, the distances and/or angles of the PAP mask facial feature vector are expressed in ranges. One skilled in the art appreciates that an exact match between a patient's facial feature vector and the PAP mask facial feature vector is at best problematic. However, when the distances and/or angles of the PAP mask facial feature vector are expressed as a range, then the probability of identifying a suitable candidate PAP mask increases to a point that it is highly likely that a suitable PAP mask may be identified for the patient.

### Bayesian Iridocorneal Scale Estimation

The anatomy of the face varies from individual to individual, including interpupillary distance, nasal width, nasal bridge height, etc. Most of these measurements are highly correlated with age, weight, gender, and height. However, in adults, with the exception of rare medical conditions, the visible dimensions of the iris of the eye, which are determined by the dimensions of the cornea and lens, are similar for all adult humans. Specifically, the horizontal white-to-white diameter of the iris averages 11.8 mm in adult humans and is uncorrelated or loosely correlated with age, weight, and other factors.

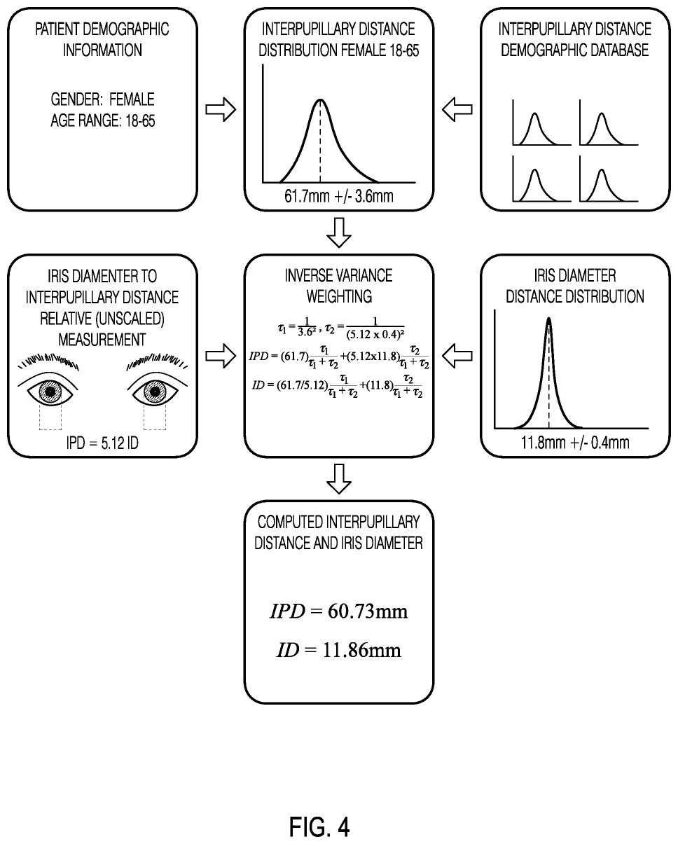

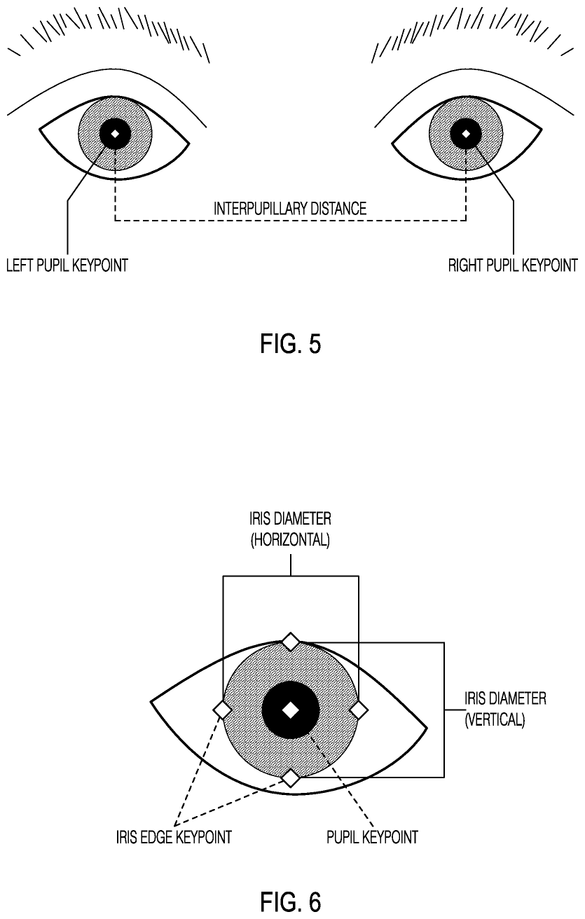

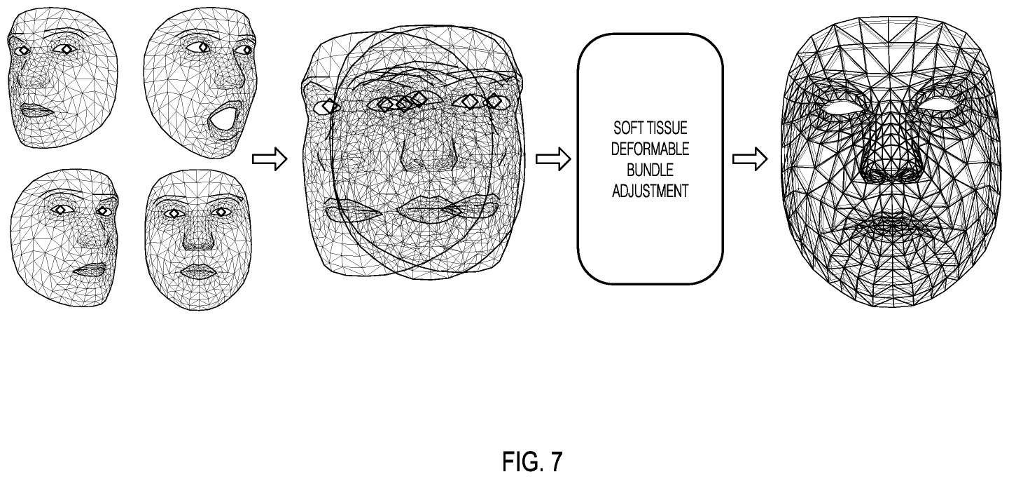

In the field of computer vision photogrammetry, when a 3D reconstruction or any set of 3D points are determined by geometric means from two-dimensional images, the resulting 3D measurements are of unknown scale. Some additional information must be inferred from the scene or from metadata in order to establish scale in known units of meters or inches, etc. The invention accomplishes this by assuming that the iris is perfectly circular and that its diameter is a random variable distributed normally with a value of 11.8+/−0.4 millimeters. This assumption is paired with an assumption about the prior distribution of interpupillary distance based on demographic factors (for example, 61.7+/−3.6 mm may be used as the expected distribution of interpupillary distance in adult females, see FIG. 4). Using the face keypoints detected by the convolutional network (see FIG. 5), a ratio or relative size relationship between the two values (interpupillary distance and iris diameter) is established. From this relationship, a Bayesian inference is used to produce the most-likely estimate for both values, by the method of inverse variance weighting. (See FIG. 4).

The ratio between the iris diameter and interpupillary distance can be determined by processing of specific keypoints extracted from 2D images of the face. Specific keypoints required are the left and right horizontal edges of each iris (for measuring iris diameter, see FIG. 5), and the center of each pupil (for measuring interpupillary distance, see FIG. 4). The keypoints are extracted via the same convolutional neural network as described in the previous section.

Once an absolute scale in millimeters has been established for interpupillary distance, this scale is used to convert all distances between keypoints into units of millimeters. The resulting absolute distances are used as input to all further mask selection and sizing processes in the system.

### Soft Tissue Deformable Bundle Adjustment

In the field of computer vision, the process of photogrammetry involves stitching together multiple 2-dimensional images into a single set of 3-dimensional points or vectors which represent a 3D object or scene. The typical final step in the process of photogrammetry-based 3D reconstruction is bundle adjustment, in which a least-squares optimization is solved to adjust the precise positions of all 3D points in the scene along with the positions and angles from which each image was captured. This optimization works by minimizing reprojection error, which is a measure of difference between predicted and observed positions of each reconstructed 3D point.

When applying photogrammetry to the human face for purposes of measurement, a common source of error is the movement of the deformable parts of the face as a result of gaze change and facial expression shift over time. If a large number of images or a video stream are captured over multiple seconds, any facial motion during the capture may cause errors to accumulate, decreasing the accuracy of collected measurements.

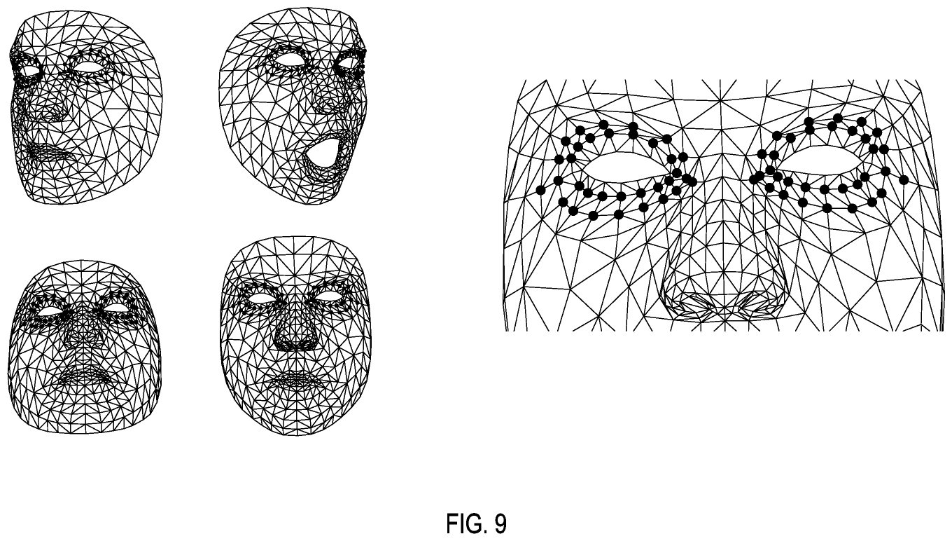

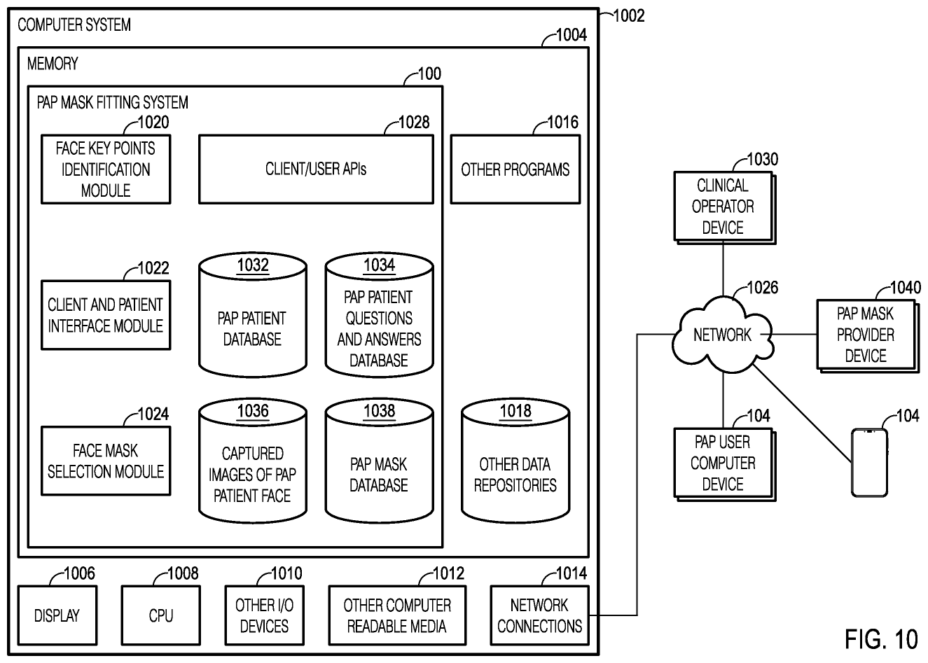

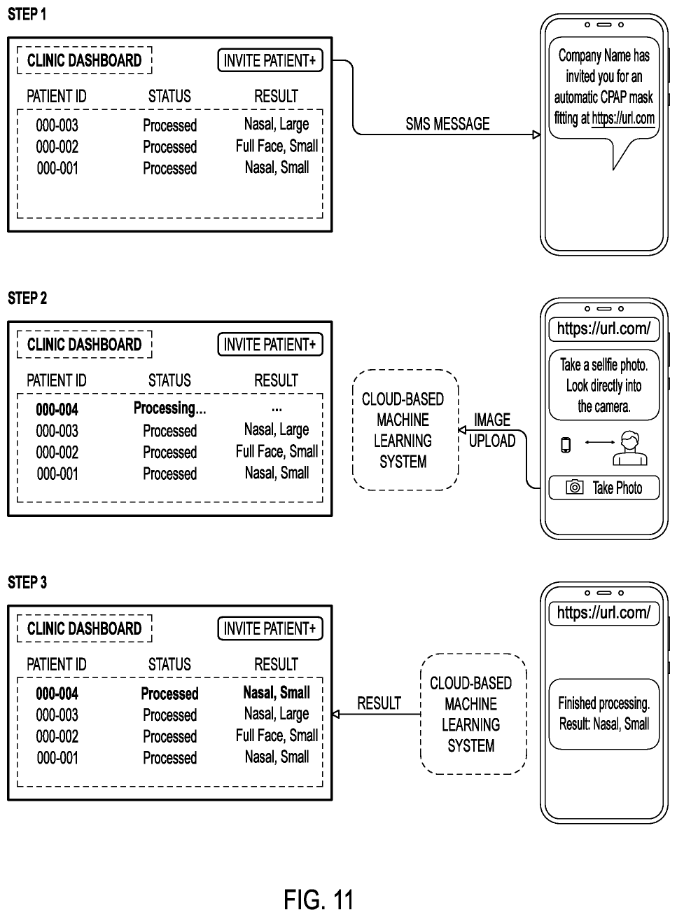

To deal with facial expression error, the bundle adjustment process can be altered with the use of a specific term accounting for deformability (the extent to which an anatomical part of the face is able to change shape, relative to the rigid underlying bony or cartilaginous tissue). After detecting keypoints on the face, the system separates the keypoints into deformable (soft tissue) and non-deformable (bony or cartilaginous tissue), and applies bundle adjustment with reprojection error weighted according to a chart of deformable vs. non-deformable face points seen in FIG. 9.

Specifically, in standard, general-purpose 3D reconstruction, reprojection error is minimized equally among all visible points, according to an equation such as the following:

$$\begin{matrix}\$\$ \\ ∖ min\_ \{ x \} ∖ sum\_ {\{ i = 1 \}}^{∧} \{ n \} ∖ sum\_ {\{ j = 1 \}}^{∧} \{ m \} {D ( Q ( x\_ \{ i j \} ) , y\_ \{ i j \} )}^{∧} 2 \\ \$\$ \\ {min}_{x} {{\sum }_{i = 1}}^{n} {{\sum }^{m}}_{j = 1} {D ( Q ( {x}_{ij} ) , {y}_{i j} )}^{2}\end{matrix}$$

where n is the number of points, m is the number of images, Q is the reprojection function, x_{ij} is the estimated position where point i should appear in image j, y_{ij} is the actual position where point i appears in image j, and D(x, y) represents Euclidean distance between points x and y.

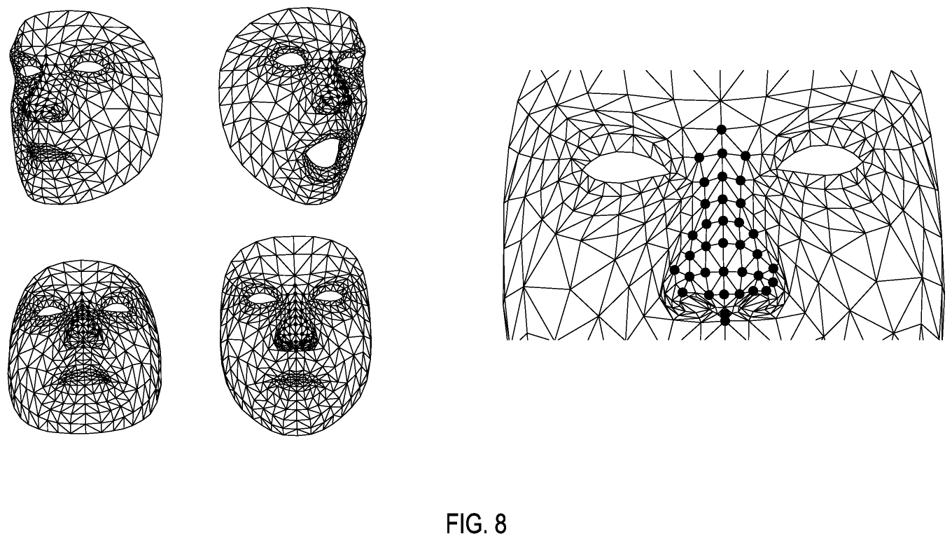

However, in the specific case of 3D reconstruction specialized for measurement of human facial anatomy, an improved reconstruction can be obtained by replacing the standard reprojection error with a Deformable Anatomy Aware reprojection error, as follows:

$$\begin{matrix}∖ min\_ \{ x \} ∖ sum\_ {\{ i = 1 \}}^{∧} \{ n \} ∖ sum\_ {\{ j = 1 \}}^{∧} \{ m \} W\_ \{ i \} {D ( Q ( x\_ \{ i j \} ) , y\_ \{ i j \} )}^{∧} 2 \\ {min}_{x} {{\sum }_{i = 1}}^{n} {{\sum }^{m}}_{j = 1} {W}_{i} {D ( Q ( {x}_{ij} ) , {y}_{i j} )}^{2}\end{matrix}$$

Where W_i is a weighting factor for each point i based on the deformability of that part of the face. Specifically, keypoints on the lips, mouth, and jaw have values W_i=0 indicating maximum deformability. Keypoints in the infraorbital area between the eyebrow and zygomatic bone, but not including the eye or eyelid, have value W_i=0.5 indicating partial deformability (see FIG. 8). Keypoints in the region from the nares and tip of the nose to the nasion point have values $W_i=1$ indicating maximum rigidity (see FIG. 9).

In this way the relative positions of the camera and the face are estimated by the alignment of the stiff, bony or cartilaginous parts of the face, without interference from movement of the deformable parts of the mouth and jaw. After completion of the bundle adjustment, the resulting keypoints can be used as described in FIG. 3 for computation of distances and input to a supervised classifier or rules-based system for mask selection.

Using any of the methods described above, the output can include a written specification for a PAP mask listing the dimensions. Another output could be a three-dimensional rendition of the PAP mask with the specific dimensions for the mask. The drawing is then used to produce tools that are used to mold the mask into a three-dimensional shape.

Additionally, the three-dimensional rendition is used to produce a three-dimensional prototype of the mask via a three-dimensional printer. The mask dimensions can be electronically communicated to the printer which then prints the prototype. The prototype could be outfitted into a complete PAP mask. The completed prototype is then sent to the patient for a fit assessment.

Alternative embodiments may use other processes and/or systems for measuring a patient's face during implementation of a PAP mask fitting system 100. Various neural network types may be used in alternative embodiments without departing substantially from the scope of this disclosure, and are intended to be included herein as alternative embodiments protected by the claims herein.

Various embodiments may employ alternative, or additional, types of communication systems and analysis systems to allow clinicians and/or patients to send links to access various data and/or to receive results data. Such non-limiting features are intended to be included herein as alternative embodiments protected by the claims herein.

FIG. 4 is a block diagram showing additional detail of an example PAP mask fitting system implemented as an example computing system 402 that may be used to practice embodiments of PAP mask fitting system 100 described herein. Note that one or more general purpose virtual or physical computing systems suitably instructed or a special purpose computing system may be used to implement a PAP mask fitting system 100. Further, the PAP mask fitting system 100 may be implemented in software, hardware, firmware, or in some combination to achieve the capabilities described herein.

Note that one or more general purpose or special purpose computing systems/devices may be used to implement the described techniques. However, just because it is possible to implement the PAP mask fitting system 100 on a general purpose computing system does not mean that the techniques themselves or the operations required to implement the techniques are conventional or well known.

The computing system 402 may comprise one or more server and/or client computing systems and may span distributed locations. In addition, each block shown may represent one or more such blocks as appropriate to a specific embodiment or may be combined with other blocks. Moreover, the various blocks of the PAP mask fitting system 100 may physically reside on one or more machines, which use standard (e.g., TCP/IP) or proprietary interprocess communication mechanisms to communicate with each other.

In the embodiment shown, computer system 402 comprises a computer memory (“memory”) 404, a display 406, one or more Central Processing Units (“CPU”) 408, Input/Output devices 410 (e.g., keyboard, mouse, CRT or LCD display, etc.), other computer-readable media 412, and one or more network connections 414. The PAP mask fitting system 100 is shown residing in memory 404. In other embodiments, some portion of the contents, some of, or all of the components of the PAP mask fitting system 100 may be stored on and/or transmitted over the other computer-readable media 412. The components of the PAP mask fitting system 100 preferably execute on one or more CPUs 408 and manage the identification of candidate PAP masks based on the patient's facial feature vector determined from the image data of the patient's face, as described herein. Other code or programs 416 and potentially other data repositories, such as data repository 418, also reside in the memory 404, and preferably execute on one or more CPUs 408 to perform other tasks. Of note, one or more of the components in FIG. 4 may not be present in any specific implementation. For example, some embodiments embedded in other software may not provide means for user input or display.

In a typical embodiment, the PAP mask fitting system 100 includes one or more face keypoints identification module 420, a client and patient interface module 422, and a face mask selection module 424. In at least some embodiments, one of more of these modules 420, 422, 424 may be provided external to the computer system 402 and is available, potentially, over one or more networks 426.

The client and patient interface module 422 is configured to facilitate establishment of a communication link between the computer system 402, the patient's electronic device 104 and the clinical operator device. Information received about the patient is stored into the PAP patient database 432.

During the initial PAP mask fitting process, the patient is asked a series of medical health questions. The client and patient interface module 422 stored the questions and answers into the PAP patient questions and answers database 434.

The client and patient interface module 433 is also configured to facilitate receiving the 2D image of the patient's face. The client and patient interface module 422 then stores the received 2D image data into the captured images of PAP patient faces database 439.

The face keypoints identification module 420 is configured to determine the face keypoints and the resultant facial feature vector as described herein. A facial feature vector is a sequence of numbers that describe measurable properties of an object, wherein each vector is a mathematical representation of a direction and a magnitude (length). Alternative embodiments may employ any suitable form of expressing a facial feature vector now known or later developed. Once determined, the patient's face keypoints and the associated facial feature vector may be stored into the PAP patient database 432.

Information about the available PAP masks is received from the PAP mask provider device 440 in an example embodiment. In some embodiments, the face mask selection module 424 harvests information about the various available PAP masks and the associated PAP mask facial feature vectors from the various manufacturers and/or vendors of PAP masks. This information is stored in the PAP mask database 438.

Once the patients facial feature vector has been determined, the face mask selection module 424 compares the patient's unique facial feature vector with the PAP mask facial feature vectors for all of the available PAP masks. Those PAP masks have a PAP mask facial feature vector that corresponds with (or is compatible with) the patient's face keypoint are identified as candidate PAP masks.

As noted hereinabove, one of the later processes is to apply a set of predetermined rules based on clinical knowledge, which may augment, or potentially override, the machine learning output. The predetermined rules may be manually applied by a clinician. Alternatively, a neural module or other suitable module may apply the predetermined rules as part of the PAP mask fitting process being performed by the PAP mask fitting system 100. For example, but not limited to, if a patient has claustrophobia, then the PAP mask fitting system 100 would not recommend a full-face style mask. Given the recommended PAP mask type/size (e.g. Nasal Mask, Medium), and based on operator preferences and availability of supplies, one or more specific models of CPAP mask (e.g. Fisher & Paykel Eson 2 with Medium headgear and Medium cushion) are identified and are then output to the patient for their consideration.

In some embodiments, the supervised machine learning classifier may employ a classification system using a probabilistic graphical model module 442. The probabilistic graphical model module 442 may represent supervised machine learning output, hand-written clinical rules, inventory preferences, and other relevant factors as changes in probability, which can be merged together to produce one final answer. Other and/or different modules may be implemented. In addition, the modules 420, 422, 424, 442 may interact via a network 426 with application or client code application program interfaces (APIs) 428 that facilitate communication with remote components, such as one or more clinical operator devices 430, such as purveyors of patient health and insurance account information stored in PAP Patient database 432. Also, of note, the PAP Patient database 432 may be provided external to the computer system 402 as well, for example in a WWW knowledge base accessible over one or more networks 426. In some embodiments, one or more of the modules 420, 422, 424, 442 may be merged together and/or merged with other modules.

In an example embodiment, components/modules of the PAP mask fitting system 100 are implemented using standard programming techniques. The module may represent x, y, z are all represented. For example, the PAP mask fitting system 100 may be implemented as a “native” executable running on the CPU 103, along with one or more static or dynamic libraries. In other embodiments, the PAP mask fitting system 100 may be implemented as instructions processed by a virtual machine. In general, a range of programming languages known in the art may be employed for implementing such example embodiments, including representative implementations of various programming language paradigms, including but not limited to, object-oriented (e.g., Java, C++, C#, Visual Basic.NET, Smalltalk, and the like), functional (e.g., ML, Lisp, Scheme, and the like), procedural (e.g., C, Pascal, Ada, Modula, and the like), scripting (e.g., Perl, Ruby, Python, JavaScript, VBScript, and the like), and declarative (e.g., SQL, Prolog, and the like).

The embodiments described above may also use well-known or proprietary, synchronous or asynchronous client-server computing techniques. Also, the various components may be implemented using more monolithic programming techniques, for example, as an executable running on a single CPU computer system, or alternatively decomposed using a variety of structuring techniques known in the art, including but not limited to, multiprogramming, multithreading, client-server, or peer-to-peer, running on one or more computer systems each having one or more CPUs. Some embodiments may execute concurrently and asynchronously and communicate using message passing techniques. Equivalent synchronous embodiments are also supported.

In addition, programming interfaces to the data stored as part of PAP mask fitting system 100 (e.g., in the data repositories 432, 434, 436, 438) can be available by standard mechanisms such as through C, C++, C#, and Java APIs; libraries for accessing files, databases, or other data repositories; through scripting languages such as XML; or through Web servers, FTP servers, or other types of servers providing access to stored data. The data repositories 432, 434, 436, 438 may be implemented as one or more database systems, file systems, or any other technique for storing such information, or any combination of the above, including implementations using distributed computing techniques.

Also the example PAP mask fitting system 100 may be implemented in a distributed environment comprising multiple, even heterogeneous, computer systems and networks. Different configurations and locations of programs and data are contemplated for use with techniques of described herein. In addition, the [server and/or client] may be physical or virtual computing systems and may reside on the same physical system. Also, one or more of the modules may themselves be distributed, pooled or otherwise grouped, such as for load balancing, reliability or security reasons. A variety of distributed computing techniques are appropriate for implementing the components of the illustrated embodiments in a distributed manner including but not limited to TCP/IP sockets, RPC, RMI, HTTP, Web Services (XML-RPC, JAX-RPC, SOAP, etc.) and the like. Other variations are possible. Also, other functionality could be provided by each component/module, or existing functionality could be distributed amongst the components/modules in different ways, yet still achieve the functions of a PAP mask fitting system 100.

Furthermore, in some embodiments, some or all of the components of the PAP mask fitting system 100 may be implemented or provided in other manners, such as at least partially in firmware and/or hardware, including, but not limited to one or more application-specific integrated circuits (ASICs), standard integrated circuits, controllers executing appropriate instructions, and including microcontrollers and/or embedded controllers, field-programmable gate arrays (FPGAs), complex programmable logic devices (CPLDs), and the like. Some or all of the system components and/or data structures may also be stored as contents (e.g., as executable or other machine-readable software instructions or structured data) on a computer-readable medium (e.g., a hard disk; memory; network; other computer-readable medium; or other portable media article to be read by an appropriate drive or via an appropriate connection, such as a DVD or flash memory device) to enable the computer-readable medium to execute or otherwise use or provide the contents to perform at least some of the described techniques. Some or all of the components and/or data structures may be stored on tangible, non-transitory storage mediums. Some or all of the system components and data structures may also be stored as data signals (e.g., by being encoded as part of a carrier wave or included as part of an analog or digital propagated signal) on a variety of computer-readable transmission mediums, which are then transmitted, including across wireless-based and wired/cable-based mediums, and may take a variety of forms (e.g., as part of a single or multiplexed analog signal, or as multiple discrete digital packets or frames). Such computer program products may also take other forms in other embodiments. Accordingly, embodiments of this disclosure may be practiced with other computer system configurations.

It should be emphasized that the above-described embodiments of the PAP mask fitting system 100 are merely possible examples of implementations of the invention. Many variations and modifications may be made to the above-described embodiments. All such modifications and variations are intended to be included herein within the scope of this disclosure and protected by the following claims.

Furthermore, the disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower, or equal in scope to the original claims, are to be considered within the subject matter of the inventions described herein.

Therefore, having thus described the invention, at least the following is claimed:

1. A method of fitting a positive airway pressure (PAP) mask used by a patient, comprising:
receiving a two-dimensional (2D) image of the PAP patient's face;
identifying a plurality of three-dimensional (3D) face keypoints from the 2D image of the PAP patient;
computing a plurality of vectors between pairs of selected 3D face keypoints,
wherein the vector mathematically represents a distance and angle between paired of selected 3D face keypoints, and
wherein the plurality of vectors define a facial feature vector of the patient;
comparing the patient's facial feature vector with a corresponding plurality of predefined PAP mask face keypoint vectors for a plurality of available PAP masks; and
identifying a candidate PAP mask from the plurality of available PAP masks that has its corresponding PAP mask facial feature vector matches the determined patient's facial feature vector; or using the face keypoint vectors to produce a three-dimensional rendition of a PAP mask; and printing the rendition of the PAP mask using a three-dimensional printer.

2. The method of claim 1, further comprising:
Using iridocorneal diameter and interpupillary distance to estimate an absolute distance between said keypoints; where said absolute distance is used as input for PAP mask selection and sizing.

3. The method of claim 2, further comprising:
Performing a soft tissue bundle adjustment.

3. The method of claim 3, further comprising:
Communicating a PAP mask recommendation to an electronic device of the PAP patient, wherein the PAP mask recommendation specifies at least a manufacturer of the candidate PAP mask and a size of the candidate PAP mask; and
communicating with the PAP mask recommendation additional information indicating to the PAP patient where the recommended candidate PAP mask can be obtained.

4. The method of claim 3, wherein after communicating the PAP mask recommendation to the PAP patient, the method further comprising:
communicating a follow up questionnaire to the PAP patient, wherein the follow up questionnaire asks questions pertaining to the patient's satisfaction of the candidate PAP mask identified in the PAP mask recommendation; and
modifying the PAP mask recommendation based upon the received answers to the follow up questionnaire.

5. The method of claim 3, wherein prior to identifying the candidate PAP mask from the plurality of available PAP masks, the method further comprising:
communicating to the electronic device of the PAP patient a set of medical questions to be answered by the PAP patient:
receiving answers to the set of medical questions from the electronic device of the PAP patient; and
modifying identification of the candidate PAP mask based upon the received answers to the set of medical questions.

6. The method of claim 5, further comprising:
determining from the answers to the set of medical questions whether the PAP patient is claustrophobic; and

7. The method of claim 3, wherein prior to receiving the 2D image, the method further comprising:
communicating instructions to an electronic device to the PAP patient,
wherein the communicated instructions specify procedures to the PAP patient pertaining to a capture of the 2D image of their face.

8. The method of claim 7, wherein communicating the instructions further comprises:
communicating a short message service (SMS) text message specifying the instructions to a cellular phone of the PAP patient,
wherein the capture 2D image is captured by the PAP patient with an image capture device on their cellular phone.

9. The method of claim 3, wherein after receiving the 2D image of the PAP patient, the method further comprising:
converting the image data of the 2D image of the PAP patient to an uncompressed image data format;
cropping the uncompressed image data so that an image of the PAP patient's face occupies a predefined amount of the total uncompressed image data; and
scaling the cropped uncompressed image data so that the face of the PAP patient is a predefined size that corresponds to standard facial size that fits each of the plurality of available PAP masks,
wherein the plurality of vectors between pairs of selected 3D face keypoints are computed from the scaled and cropped uncompressed image data.

10. The method of claim 9, further comprising:
aligning the image of the face of the PAP patient with a predefined standard alignment,
wherein the plurality of vectors between pairs of selected 3D face keypoints are computed based on the image data with the aligned face of the PAP patient.

11. The method of claim 3, wherein identifying the PAP mask from the plurality of available PAP masks comprises:
identifying a size of the candidate PAP mask.

12. The method of claim 3, wherein identifying the PAP mask from the plurality of available PAP masks comprises:
identifying a type of the candidate PAP mask from among the plurality of different types of PAP masks.

13. The method of claim 3, wherein the PAP patient is a first PAP patient, the method further comprising:
storing the plurality of vectors between pairs of selected 3D face keypoints into the database with an association with the first PAP patient, wherein information corresponding to a plurality of vectors between pairs of selected 3D face keypoints of the second PAP patient is compared to the plurality of vectors between pairs of selected 3D face keypoints of the first PAP patient in the learning process by a machine learning classifier;
comparing a candidate PAP mask for the second PAP patient with the candidate PAP mask identified for the first PAP patient; and
verifying the candidate PAP mask for the second PAP patient when plurality of vectors between pairs of selected 3D face keypoints is the same as the plurality of vectors between pairs of selected 3D face keypoints of the first PAP patient.

14. The method of claim 3, wherein the PAP patient is a first PAP patient, the method further comprising:
storing the plurality of identified 3D face keypoints in a database with an association with the first PAP patient;
comparing a plurality of identified 3D face keypoints identified in the 2D image of a second PAP patient to the stored plurality of identified 3D face keypoints of the first PAP patient in a learning process using a machine learning classifier, and
comparing a candidate PAP mask for the second PAP patient with the candidate PAP mask identified for the first PAP patient; and
verifying the candidate PAP mask for the second PAP patient when the candidate plurality of identified 3D face keypoints of the second PAP patient is the same as the identified 3D face keypoints of the first PAP patient.

15. The method of claim 3, wherein the received 2D image of the PAP patient is a first 2D image of the PAP patient, and wherein the plurality of vectors between pairs of selected 3D face keypoints are a first plurality of vectors between pairs of selected 3D face keypoints, the method further comprising:
receiving a second 2D image of the PAP patient's face;
identifying a second plurality of 3D face keypoints from the second 2D image of the PAP patient;
computing a second plurality of vectors between pairs of selected plurality of 3D face keypoints;
normalizing the second plurality of vectors between pairs of selected 3D face keypoints with the first plurality of vectors between pairs of selected 3D face keypoints determined from the first 2D image of the PAP patient; and
averaging each of the first and second plurality of vectors between pairs of selected 3D face keypoints to compute an average plurality of vectors between pairs of selected 3D face keypoints,
wherein the candidate PAP mask is identified based on the averaged plurality of vectors between pairs of selected 3D face keypoints.

16. The method of claim 15, wherein the first 2D image and the second 2D image of the PAP patient are in a video clip taken on the patient's face.

17. The method of claim 3, wherein the received 2D image of the PAP patient is a first 2D image of the PAP patient, and wherein the plurality of vectors between pairs of selected 3D face keypoints are a first plurality of vectors between pairs of selected 3D face keypoints, the method further comprising:
receiving a second 2D image of the PAP patient's face;
identifying a plurality of 3D face keypoints from the second 2D image of the PAP patient;
normalizing the second plurality of vectors between pairs of selected 3D face keypoints with the first plurality of identified 3D face keypoints determined from the first 2D image of the PAP patient;
comparing the second plurality of identified 3D face keypoints with the first plurality of identified 3D face keypoints; and
averaging each of the first and second plurality of plurality of identified 3D face keypoints to compute an average location for the plurality of identified 3D face keypoints,
wherein the plurality of vectors between pairs of selected 3D face keypoints are computed based on the averaged location for the plurality of identified 3D face keypoints.

18. The method of claim 3, further comprising:
receiving information about the plurality of available PAP masks from the makers of the plurality of available PAP masks, wherein the information specifies at least the plurality of vectors between pairs of selected 3D face keypoints for each one of the plurality of identified 3D face keypoints;
storing the received information about the plurality of available PAP masks in a database; and
accessing the stored information about the plurality of available PAP masks when the plurality of vectors between pairs of selected 3D face keypoints computed from the received 3D image of the PAP patient are compared with the corresponding plurality of PAP mask vectors for the plurality of available PAP masks.

19. The method of claim 3, further comprising:
receiving information about the plurality of available PAP masks from the makers of the plurality of available PAP masks;
computing the plurality of vectors between pairs of selected 3D face keypoints for each one of the plurality of available PAP masks based on the received information;
storing the computed plurality of vectors between pairs of selected 3D face keypoints in a database;
accessing the stored computed plurality of vectors between pairs of selected 3D face keypoints of the available PAP masks when the plurality of vectors between pairs of selected 3D face keypoints computed from the received 3D image of the PAP patient are compared with the corresponding plurality of PAP mask vectors for the plurality of available PAP masks.
